(19) United States
(12) Patent Application Publication
(10) Pub. No.: US 2006/0198888 A1
(43) Pub. Date: Sep. 7, 2006

(54) Tolperisone-containing pharmaceutical preparation with controllable active-substance release for oral administration

(75) Inventors: Angelika Bodenteich, Wien (AT); Eberhard Pirich, Wien (AT); Josef Bockmann, Modling (AT); Werner Frantsits, Wien (AT)

Correspondence Address: ROBINS & PASTERNAK LLP, 1731 EMBARCADERO ROAD, SUITE 230, PALO ALTO, CA, 94303, US

(73) Assignee: Sanochemia Pharmazeutika AG

(21) Appl. No.: 11/356,405

(22) Filed: Feb. 16, 2006

Related U.S. Application Data
(63) Continuation of application No. 10/932,043, filed on Sep. 2, 2004, now abandoned.

(30) Foreign Application Priority Data
Mar. 5, 2004 (AT) 386/2004

(51) Int. Cl. A61K 31/445; A61K 9/22

(52) U.S. Cl. 424/468; 514/317

(57) ABSTRACT

The invention relates to a tolperisone-containing pharmaceutical preparation with controllable active-substance release for oral administration, characterised in that the active substance tolperisone and/or a pharmaceutical salt thereof is embedded in a pharmaceutically compatible material. By selecting the pharmaceutically compatible materials in the preparation and accordingly in the coating of a tablet or granule, a specific release of active substance can be adjusted which is matched to the special genotype in the metabolisation of tolperisone. At the same time, as a result of the very uniform and persistent release of tolperisone, the in-vivo inversion of enantiomerically pure tolperisone that is known from the art can be adjusted in favour of the R(−) tolperisone which is prominent in muscle-relaxing therapy.

This application is a continuation of U.S. Ser. No. 10/932,043, filed Sep. 2, 2004, and claims priority of Austrian Patent Application No. A 386/2004, filed Mar. 5, 2004, the contents of each of which are hereby incorporated herein by reference.

The invention relates to a tolperisone-containing pharmaceutical preparation with controllable active-substance release for oral administration.

Tolperisone is the international non-proprietary name for the muscle relaxant (RS)-2,4′ dimethyl-3-piperidinopropiophenone. The enantiomeric separation of tolperisone present as racemate is described in JP-A-53-40779. In this case, enantiomerically pure tolperisone is formed by diastereomer formation from racemic tolperisone and enantiomerically pure acetylphenylglycine salts. The diastereomers were separated by selective precipitation so that after separation of the acetylphenylglycine groups both R(−) and S(+) tolperisone was obtained in enantiomerically pure form.

Zsila et al. (Chirality 12: 720-726, 2000) have also dealt with the stereochemistry of tolperisone and established that the absolute configuration of (−) tolperisone corresponds to an R-configuration. This has also been confirmed by a monocrystal analysis which has shown that (+) tolperisone corresponds to the S configuration.

The pharmacological effect of the two enantiomers was also discussed in JP-A-53-40779. The pharmacological investigations describe a muscle-relaxing effect of d-tolperisone and a vaso- or bronchodilatory effect of 1-tolperisone.

Despite the proven pharmaceutical efficacy of enantiomerically pure tolperisone and its pharmaceutically compatible salts, the oral administration is problematical insofar as the desired effect diminishes rapidly and the patient must therefore take tolperisone-containing preparations several times a day whereby the gastro-intestinal tract of the patient can sometimes be damaged.

Tolperisone is metabolised relatively rapidly in the body wherein the enzyme CYP2D6 substantially influences the type and duration of the metabolism. Four different genotypes have been determined for this enzyme, namely “poor metabolisers” (approximately 7% of the population), “ultrafast metabolisers” (approximately 3%), “extensive metabolisers” (approximately 45%) and “intermediate metabolisers” (approximately 45%). The last two groups mentioned are only genotypically distinguishable but not phenotypically distinguishable. Especially in the group of “poor metabolisers”, there is a risk of toxicity since tolperisone is only converted very slowly.

In order to nevertheless achieve the desired long-term effect, it was proposed in JP-A-3277239 to develop transdermal formulations. However, practice shows that transdermal transport of medicinal products is limited especially with regard to dosage since unit doses of max. 150 mg can only be administered transdermally whereby an effective therapy is not yet established.

WO-A-00/59508 describes tolperisone-containing formulations which can be administered orally but do not have the disadvantages of the known tolperisone preparations which can be administered orally. In this case, an attempt was made to utilise the delayed effect of tolperisone insofar as the release behaviour of tolperisone should also be influenced by a defined selection of the enantiomeric ratio of R(−) to S(+) tolperisone. The adjustment of a defined enantiomeric ratio by chemical reaction is occasionally expensive and besides need not result in the desired pharmaceutical effect. Thus, in their article “Determination of Tolperisone Enantiomers in Plasma and Their Disposition in Rats” (Chem. Pharm. Bull. 4001, 272-274, Vol. 40 (1992)), Teruyoshi Yokoyama et al. have shown that an in-vivo inversion can be detected when using enantiomerically pure tolperisone. This means that through this in-vivo inversion enantiomerically pure S(+) tolperisone is converted into R(−) tolperisone to an extent of up to 20% or enantiomerically pure R(−) tolperisone is converted into S(+) tolperisone in a fraction up to 20%. This in-vivo inversion can reduce the desired pharmaceutical effect and also casts into question the use of enantiomerically pure tolperisone.

The object of the invention is to influence this in-vivo inversion by a particular, orally administrable pharmaceutical formulation wherein at the same time the controllability of the active substance release should also be modulated with the objective of long-term therapy.

According to the invention, a tolperisone-containing pharmaceutical preparation of the type specified initially is proposed, comprising the active substance tolperisone and/or a pharmaceutically compatible salt thereof embedded in a pharmaceutically compatible material.

Preferred and advantageous embodiments of the preparation according to the invention are the subject matter of the dependent claims.

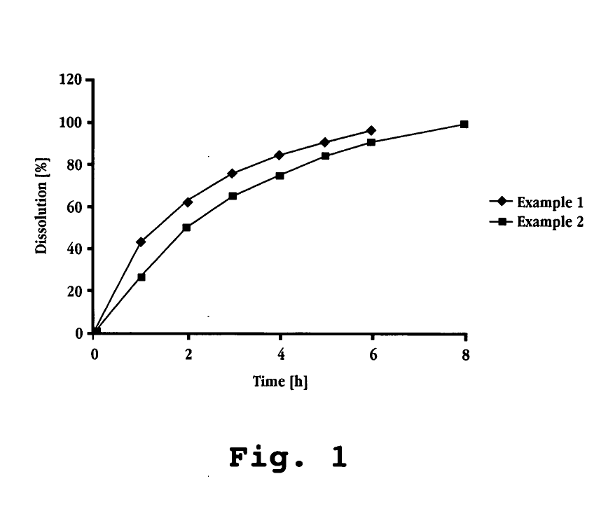

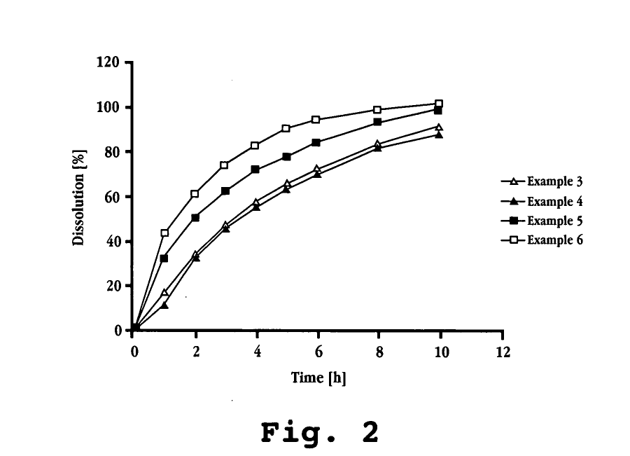

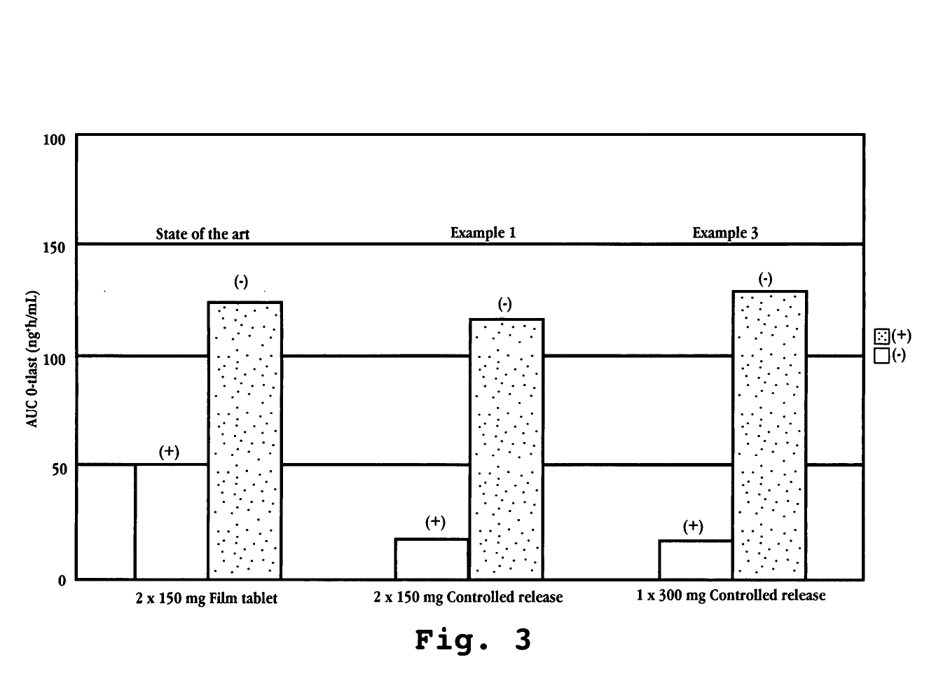

The invention is now explained in detail with reference to exemplary embodiments and with reference to FIGS. 1 and 2 which show the release profiles of preparations according to the examples, and with reference to FIG. 3 relating to the in-vivo fraction of S(+) or R(−) tolperisone.

### EXAMPLE 1

Tolperisone hydrochloride is granulated with a solution consisting of Eudragit RS in butanone in a mixer. Eudragit S and Eudragit L are then mixed in homogeneously, the mixture is dried and sieved. The sieved granular material is then mixed with tabletting excipients and tabletted. Tablets having a diameter of 8 mm and a weight of 190 mg are pressed.

The tablets are then coated with a film material consisting of Eudragit L, colouring agents and other excipients which are dissolved in butanol.

| | |
|---|---|
| Tolperisone hydrochloride | 150.00 |
| Eudragit RS | 1.88 |
| Eudragit L | 14.24 |
| Eudragit S | 10.50 |
| Aerosil | 1.80 |
| Stearic acid | 1.80 |
| Glycerol dibehenate | 7.50 |
| Iron oxide colouring agent | 0.08 |
| Titanium dioxide | 4.08 |
| Talc | 6.03 |
| Polyethylene glycol | 1.02 |
| Dimethylpolysiloxane | 0.05 |

It can be seen from FIG. 1 that the preparation according to Example 1 shows a relatively rapid release of active substance, namely approximately 60% in two hours and approximately 85% in four hours. After stress storage at 40° C. and 75% humidity, no degradation of the active substance is observed over a period of three months. All tested by-products are below the limit value of <0.2%.

### EXAMPLE 2

In this example the manufacture and composition of a 200 mg tolperisone hydrochloride formulation with average release rate are described. For the manufacture, tolperisone hydrochloride is granulated with a solution consisting of Eudragit RS in butanone. Eudragit S and Eudragit L are then mixed in homogeneously. The mixture is dried and sieved. After the required tabletting excipients have been homogeneously mixed in, tablets having a diameter of 9 mm and a weight of 250 mg are pressed. These tablets are then film-coated with a solution consisting of Eudragit L, colouring agent and other excipients which are dissolved in butanol.

| | |
|---|---|
| Tolperisone hydrochloride | 200.00 |
| Eudragit RS | 2.50 |
| Eudragit L | 16.60 |
| Eudragit S | 12.85 |
| Aerosil | 2.40 |
| Stearic acid | 2.40 |
| Glycerol dibehenate | 2.40 |
| Iron oxide colouring agent | 0.08 |
| Titanium dioxide | 4.08 |
| Talc | 10.02 |
| Polyethylene glycol | 1.02 |
| Dimethylpolysiloxane | 0.05 |

The tolperisone-200 mg “controlled release” formulation according to the example shows a release of active substance of approximately 50% in 2 hours and approximately 80% in 5 hours. As can be seen from FIG. 1, this is a comparatively moderate release rate.

### EXAMPLE 3

This example describes the manufacture of a 300 mg tolperisone “controlled release” formulation with constant long-term retardation. Manufacture takes place in a high-speed mixer. Tolperisone is granulated with a granulating solution of Eudragit RS dissolved in butanone. Eudragit L and Eudragit S are then added and dried after homogeneous mixing. The granular material obtained is then mixed homogeneously with tabletting excipients and then pressed into tablets having a diameter of 10 mm and a weight of 380 mg. The tablets are film-coated using a solution of Eudragit RS, colouring agent and other excipients in butanone.

| | |
|---|---|
| Tolperisone hydrochloride | 300.00 |
| Eudragit RS | 11.60 |
| Eudragit L | 21.00 |
| Eudragit S | 21.00 |
| Aerosil | 3.60 |
| Stearic acid | 3.60 |
| Glycerol dibehenate | 15.00 |
| Iron oxide colouring agent | 1.26 |
| Titanium dioxide | 6.28 |
| Talc | 14.14 |
| Dimethylpolysiloxane | 0.07 |
| Magnesium stearate | 0.50 |

As can be seen from FIG. 2, in the formulation according to the example, the release of active substance is significantly delayed. This means that 50% of the active substance is released after approximately 3 hours and 80% after approximately 7.5 hours. The stability stress test at 40° C. and 75% humidity over 3 months shows tolperisone in a stable form and a fraction of degradation products of <0.02%.

### EXAMPLE 4

This example describes a tolperisone hydrochloride “controlled release” formulation with 300 mg active substance and a very strongly retarded release profile. Manufacture takes place by forming a paste of tolperisone in a pharmaceutical mixer whilst adding a solution consisting of Eudragit RS dissolved in acetone and isopropanol, with Eudragit S and Eudragit L then being mixed in homogeneously. The premixed mass obtained is then dried and sieved. After adding tabletting excipients, tablets are pressed. These tablets are coated with a film consisting of Eudragit RS and colouring agent as well as further pharmaceutical excipients.

| | |
|---|---|
| Tolperisone hydrochloride | 300.00 |
| Eudragit RS | 27.60 |
| Eudragit L | 21.00 |
| Eudragit S | 21.00 |
| Aerosil | 3.60 |
| Glycerol dibehenate | 18.00 |
| Talc | 22.00 |
| Magnesium stearate | 3.60 |
| Triethyl citrate | 7.50 |

As can be seen from FIG. 2, the formulation according to the example shows a very uniform release of active substance over a long time. That is, 50% of the active substance is released in approximately 3 hours, 80% of the active substance is released in approximately 8 hours. A 100% release of active substance is expected in approximately 12 hours.

### EXAMPLE 5

Example 5 shows a tolperisone “controlled release” formulation with 300 mg of active substance and moderate release rate. The tablet core and the film are manufactured as in Example 4. However, significantly less material is applied.

| | |
|---|---|
| Tolperisone hydrochloride | 300.00 |
| Eudragit RS | 18.10 |
| Eudragit L | 21.00 |
| Eudragit S | 21.00 |
| Aerosil | 3.60 |
| Glycerol dibehenate | 18.00 |
| Talc | 18.00 |
| Magnesium stearate | 3.60 |
| Triethyl citrate | 4.50 |

FIG. 2 shows a release of active substance of 50% in approximately 2 hours and 80% release after approximately 5.5 hours. The steepness of the curve shows a somewhat faster surge at the beginning of the release and a flattening towards the end of the release of active substance.

### EXAMPLE 6

Example 6 shows a 300 mg tolperisone “controlled release” formulation with slightly delayed release. The tablet core is manufactured as in Example 4. Significantly less film material is used compared with Example 4 and 5.

| | |
|---|---|
| Tolperisone hydrochloride | 300.00 |
| Eudragit RS | 8.25 |
| Eudragit L | 21.00 |
| Eudragit S | 21.00 |
| Aerosil | 3.70 |
| Glycerol dibehenate | 18.00 |
| Talc | 14.00 |
| Magnesium stearate | 3.60 |
| Triethyl citrate | 1.50 |

FIG. 2 shows a “controlled release” with a very rapid release of active substance in the formulation according to the example. Thus, 50% of the active substance is already released after 1.3 hours, and 80% of the active substance after approximately 3.5 hours.

### EXAMPLE 7

Example 7 describes a 150 mg tolperisone-containing “controlled release” formulation with delayed release which additionally has a gastric-juice resistant coating. For its manufacture tolperisone hydrochloride is granulated with Eudragit solution and then dried. The sieved granular material is mixed with tabletting excipients and tabletted. Tablets having a diameter of 8 mm and a weight of 196 mg are pressed. The tablets are coated with a gastric-juice resistant film.

| | |
|---|---|
| Tolperisone hydrochloride | 150.00 |
| Eudragit RS | 1.88 |
| Eudragit S | 10.20 |
| Triethyl citrate | 0.69 |
| Aerosil | 1.80 |
| Stearic acid | 1.80 |
| Glycerol dibehenate | 7.70 |
| Titanium dioxide | 6.02 |
| Eudragit L | 14.24 |
| Polyethylene glycol | 1.52 |
| Dimethyl polysiloxane | 0.15 |

The film-coated tablets according to the example show none or extremely little release of active substance in gastric juice over a period of 1-2 hours. After buffering to pH 6.8 a somewhat slowed release of active substance takes place.

With reference to the release profiles according to FIGS. 1 and 2, it can be shown that the tolperisone-containing pharmaceutical preparations according to the invention of exemplary embodiments 1 and 6 show a relatively rapid release of active substance whereas the preparations according to Examples 2 and 5 show a moderate release of active substance and those according to Examples 3 and 4 yield a slow but very uniform release of the active substance. Since tolperisone is metabolised at different rates in the human body (which resulted in the classification of four different genotypes with reference to the enzyme CYP2D6 studied in this context, namely the “poor metabolizer”, the “ultrafast metabolizer”, the “extensive metabolizer” and the “intermediate metabolizer”) , as a result of its controllable release of active substance, the tolperisone-containing pharmaceutical preparation according to the invention can be matched to the particular genotype according to the rate of release. Thus, the tolperisone-containing pharmaceutical preparations manufactured according to Examples 1 and 6 can be administered to the so-called “ultrafast metabolizer” as a result of their relatively rapid release of active substance, with the formulations according to Examples 2 and 5 being administered to the “extensive” or also to the “intermediate metabolizer” since these respond to tolperisone-containing pharmaceutical preparations with a moderate release of active substance.

In the case of the genotype of the “poor metabolizer” who must be treated with the active substance tolperisone over a relatively long period in order to produce sufficient saturation of the active substance in the blood level, however it is possible to administer the pharmaceutical preparations according to Examples 3 and 4.

The delayed release of tolperisone which is achieved with the pharmaceutical preparation according to the invention can be explained insofar as the active substance tolperisone is predominantly embedded in a polymer matrix which is pharmaceutically compatible and which during the metabolisation of tolperisone allows a delayed but specific release of tolperisone as a result of the embedding in the matrix material. This release can be additionally supported by the fact that in the case of tablets, the tablet cores are additionally surrounded by a coating which delays the release of the active substance. The materials used for this coating advantageously consist of pharmaceutically compatible polymers which likewise bring about a slowed but at the same time controllable release as a result of their matrix structure. A uniform saturation of tolperisone in the blood plasma level is thereby achieved so that undesirable, so-called “overshooting peaks” in the blood plasma level accordingly can be avoided. This yields an advantageous effect in the administration of the tolperisone-containing pharmaceutical formulation according to the invention with regard to the rare “poor metabolizer” and “ultrafast metabolizer” CYP2D6 genotype groups. As a result of the specific release, a reduction in the toxicity risk and thus a reduction in the rate of side effects is obtained for the “poor metabolizer”, whereas in the case of the “ultrafast metabolizer” a more uniform and therefore improved level of action can be achieved over a longer time compared with conventional film-coated tablets. As a result of the controllable and therefore uniform release of active substance, the aforesaid genotypes are supplied with the active substance tolperisone over a longer time so that the blood plasma level is sufficiently saturated with tolperisone.

As a result of the controllable release of active substance by the tolperisone-containing pharmaceutical preparation according to the invention, the in-vivo inversion known in the art can be controlled in the direction of the desired R(−) tolperisone which is effective in muscle-relaxing therapy. In this case, the blood plasma level of patients who were treated with conventional film-coated tablets (FT tablets) shows a larger AUC (Area under the curve) with regard to R(−) tolperisone compared to the S(+) tolperisone which is undesirable in muscle-relaxing therapy.

However, if film-coated tablets such as those manufactured in accordance with Example 1 are administered, the fraction of S(+) tolperisone after in-vivo inversion is thus reduced further in contrast to the in-vivo inversion with known film-coated tablets.

The same effect was shown using preparations according to the invention such as those of Example 3, wherein the fraction of desired R(−) tolperisone could be additionally increased as a result of the particularly slow and specific release of active substance.

Thus, not only the active substance release profile can be specifically adjusted using the tolperisone-containing pharmaceutical preparation according to the invention but at the same time an optimal usage of the in-vivo inversion of enantiomerically pure tolperisone can be achieved in favour of the R(−) enantiomer required for the muscle-relaxing therapy. Accordingly, the tolperisone-containing pharmaceutical formulation according to the invention is used in muscle-relaxing therapy and in the treatment of muscle spasms of various etiology which are triggered by degenerative changes to the spine such as the cervical syndrome, lumbago, cervico-brachial syndrome and similar. However, areas of application are also found in the treatment of osteoporosis as well as arthritis of the knee and/or hip joints and in rheumatic diseases such as soft-tissue rheumatism or chronic polyarthritis. Another area of usage is in the area of treatment of fibromyalgia and in supportive therapy following work and/or sports injuries. The tolperisone-containing pharmaceutical preparation according to the invention is furthermore used in the treatment of spasticity as a result of neurological diseases. Suspensions of tolperisone granules are used with particular advantage if these are administered to children with corresponding flavour enhancers.

However, the tolperisone-containing pharmaceutical preparations according to the invention are also used in the rehabilitation treatment of strokes and in the treatment of multiple sclerosis, Parkinson's disease and climacteric symptoms.

The tolperisone-containing pharmaceutical preparations according to the invention are capable of producing long-lasting uniform levels of action. It can be deduced from recent clinical test reports that tolperisone, especially in high doses, is capable of influencing the pain memory. Under these conditions, tolperisone can also be used successfully to treat diabetic neuropathy, post-herpetic neuralgia and arthritis in Lyme disease (borreliosis).

In summary, the tolperisone-containing pharmaceutical preparation according to the invention allows a specific and controllable dosing without free active substance insofar as the active principle tolperisone is embedded in a suitable pharmaceutical carrier, preferably a polymer matrix. As a result, by selecting the materials for the matrix or coating of the tablet or granules, a release of active substance matched to the special genotype can be achieved. At the same time, as a result of the very uniform and persistent release of tolperisone, the known in-vivo inversion of enantomerically pure tolperisone can be adjusted in favour of the R(−) tolperisone relevant in muscle-relaxing therapy.

1. A tolperisone-containing pharmaceutical preparation with controllable active-substance release for oral administration, characterised in that the active substance tolperisone and/or a pharmaceutically acceptable salt thereof is embedded in a pharmaceutically compatible material.

2. The tolperisone-containing pharmaceutical preparation according to claim 1, characterised in that the pharmaceutically compatible material consists of synthetic and/or natural polymers and/or copolymers.

3. The tolperisone-containing pharmaceutical preparation according to claim 1, characterised in that the pharmaceutically compatible material is a matrix consisting of synthetic and/or natural polymers and/or copolymers.

4. The tolperisone-containing pharmaceutical preparation according to claim 1, characterised in that the pharmaceutically compatible material consists of polymers and/or copolymers selected from the group of acrylic and/or methacrylic acid esters, preferably methyl and/or ethyl (meth) acrylate as well as mixtures thereof.

5. The tolperisone-containing pharmaceutical preparation according to claim 1, characterised in that the active substance tolperisone is present in racemic form.

6. The tolperisone-containing pharmaceutical preparation according to claim 1, characterised in that the active substance tolperisone is present predominantly as a (+) enantiomer.

7. The tolperisone-containing pharmaceutical preparation according to claim 1, characterised in that the active substance tolperisone is present predominantly as a (−) enantiomer.

8. The tolperisone-containing pharmaceutical preparation according to claim 1, characterised in that the pharmaceutical formulation is a tablet which has a tablet core consisting of the active substance in the form of tolperisone and/or a pharmaceutically compatible salt thereof embedded in a pharmaceutically compatible material and a coating surrounding the tablet core which delays the release of the active substance.

9. The tolperisone-containing pharmaceutical preparation according to claim 1, characterised in that the pharmaceutical formulation is a granular material in which the granules have a core consisting of the active substance in the form of tolperisone and/or a pharmaceutically compatible salt thereof embedded in a pharmaceutically compatible material and a coating surrounding the core which delays the release of the active substance.

10. The tolperisone-containing pharmaceutical preparation according to claim 9, characterised in that the granular material is present in the form of capsules.

11. The tolperisone-containing pharmaceutical preparation according to claim 9, characterised in that the granular material is present in suspension.

12. The tolperisone-containing pharmaceutical preparation according to claim 8, characterised in that the material of the coating which delays the release of the active substance consists of pharmaceutically compatible synthetic and/or natural polymers and/or copolymers.

13. The tolperisone-containing pharmaceutical preparation according to claim 12, characterised in that the material of the coating which delays the release of the active substance is a matrix consisting of pharmaceutically compatible synthetic and/or natural polymers and/or copolymers.

14. The pharmaceutical preparation according to claim 12, characterised in that the pharmaceutically compatible polymers and/or copolymers are selected from the group of acrylic and/or (meth)acrylic acid esters, preferably methyl and ethyl (meth) acrylates as well as mixtures thereof.

15. The tolperisone-containing pharmaceutical preparation according to claim 8, characterised in that the sum of the pharmaceutical material in the core and the coating of one tablet or granule lies in a range of 5 to 50% (w/w) relative to 100% (w/w) of the tablet or granule.

16. The tolperisone-containing pharmaceutical preparation according to claim 15, characterised in that the sum of the pharmaceutical material in the core and the coating of one tablet or granule lies in a range of 10 to 20% (w/w) relative to 100% (w/w) of the tablet or granule.

17. The tolperisone-containing pharmaceutical preparation according to claim 8, characterised in that the active-substance dosage lies in a range of 50 to 600 mg, preferably 150 to 300mg.

18. The tolperisone-containing pharmaceutical preparation according to claim 8, characterised in that by selecting the pharmaceutical material for the core and/or the coating of the tablet or granule, a release of active substance is brought about in a time interval of 1 to 12, preferably 4 to 8 hours.

19. The tolperisone-containing pharmaceutical preparation according to claim 8, characterised in that by selecting the pharmaceutical material for the core and/or the coating of the tablet or granule, a release of active substance is brought about in a time interval of 5 to 24, preferably 7 to 12 hours.

20. The tolperisone-containing pharmaceutical preparation according to claim 8 for use in muscle-relaxing therapy, characterised in that by selecting the pharmaceutical material for the core and/or the coating of the tablet or granule, the in-vivo fraction of the muscle-relaxing (−) tolperisone is significantly elevated compared to the (+) tolperisone.

21. The tolperisone-containing pharmaceutical preparation according to claim 20, characterised in that the fraction of (+) tolperisone is lower than 20%, preferably lower than 17% relative to 100% in-vivo tolperisone.

22. The tolperisone-containing pharmaceutical preparation according to claim 8, characterised in that the surface of the tablet or granule is coated with a coating which delays the release of the active substance, which has the result that approximately 100% of the active substance is only released after passage through the stomach.

23. The use of a tolperisone-containing pharmaceutical preparation according to claim 1 for manufacturing a medicinal product for the treatment of diabetic neuropathy.

24. The use of a tolperisone-containing pharmaceutical preparation according to claim 1 for manufacturing a medicinal product for the treatment of post-herpetic neuralgia.

25. The use of a tolperisone-containing pharmaceutical preparation according to claim 1 for manufacturing a medicinal product for the treatment of arthritis in Lyme disease.
